(19) United States
(12) Patent Application Publication
(10) Pub. No.: US 2025/0186086 A1
(43) Pub. Date: Jun. 12, 2025

(54) LOCKING MECHANISM FOR CANNULIZED SURGICAL INSTRUMENTS

(71) Applicant: Salvus Medical LLC, Miller Place, NY (US)

(72) Inventors: Nicholas Mancini, Miller place, NY (US); Jamie Mancini, Miller place, NY (US)

(73) Assignee: Salvus Medical LLC, Miller Place, NY (US)

(21) Appl. No.: 18/846,106

(22) PCT Filed: Mar. 10, 2023
(86) PCT No.: PCT/US2023/064181

§ 371 Date: Sep. 11, 2024

Related U.S. Application Data
(60) Provisional application No. 63/319,006, filed on Mar. 11, 2022.

(51) Int. Cl. A61B 17/34

(52) U.S. Cl. CPC A61B 17/3496

(57) ABSTRACT

In accordance with at least one aspect of this disclosure, a locking mechanism for a surgical device includes one or more relatively movable telescoping tubular portions configured for movement along a longitudinal axis between a first position and a second position, the locking mechanism being associated with at least one of the relatively moveable tubular portions and being configured to selectively control the relative longitudinal movement of the one or more tubular portions between the first position and the second position.

### CROSS-REFERENCE TO RELATED APPLICATION

This is a national stage entry of International Patent Application No. PCT/US2023/064181, filed Mar. 10, 2023, which claims priority to and the benefit of U.S. Provisional Patent Application No. 63/319,006, filed Mar. 11, 2022, the entire contents of which are incorporated by reference herein.

### BACKGROUND OF THE DISCLOSURE

### Field of the Disclosure

The present invention relates to surgical instrumentation, and more particularly, to a locking mechanism for use with a cannulized surgical device to improve safety.

### Description of Related Art

Conventional Veress needles can be 12 to 15 cm long and typically include an outer cannula having of a sharp beveled needle point for cutting through tissues of the abdominal wall. A spring-loaded, inner stylet is positioned within the outer cannula. This inner stylet has a dull or blunt tip to protect any viscera from injury by the sharp, outer cannula. When penetrating through tissue, direct pressure on the beveled cutting tip pushes the blunt stylet into the shaft of the outer cannula, allowing the sharp beveled edge to penetrate the abdominal wall. Carbon dioxide gas can then be passed through the Veress needle to inflate the abdominal cavity, thus creating a pneumoperitoneum.

However, there is currently no means to lock the inner stylet in place, so as to prevent accidental penetration of surrounding tissue by the outer cannula. Thus, there remains a need in the art for a locking mechanism for cannulized surgical devices. This disclosure provides a solution for this need.

### SUMMARY OF THE INVENTION

In accordance with at least one aspect of this disclosure, a locking mechanism for a surgical device is disclosed. The surgical device has one or more relatively movable telescoping tubular portions configured for movement along a longitudinal axis between a first position and a second position. The locking mechanism is associated with at least one of the relatively moveable tubular portions and the locking mechanism is configured to selectively control the relative longitudinal movement of the one or more tubular portions between the first position and the second position.

In accordance with at least one aspect of this disclosure, a surgical device includes, a proximal handle, an elongated tubular inner cannula having a proximal portion extending from the handle and a distal end portion having a first configuration and an elongated tubular outer cannula having a proximal portion extending from the handle a distal end portion having a second configuration. The inner cannula is configured to move within the outer cannula between a first inner cannula position and a second inner cannula position. The surgical device also includes a locking mechanism operatively associated with the handle. The locking mechanism is adapted and configured to lock the inner cannula relative to the outer cannula to prevent movement of the inner cannula between the first inner cannula position and the second inner cannula position.

In embodiments, the distal end portion of the inner tubular cannula has a blunt configuration and in embodiments, the distal end portion of outer cannula has cutting edge configuration to puncture a tissue. In embodiments, the cutting edge configuration can be a beveled cutting edge configuration.

The locking mechanism is adapted and configured to lock the inner cannula in the first inner cannula position. In embodiments, when the inner cannula is in the first inner cannula position the blunt distal end portion of the inner cannula extends beyond the beveled distal end portion of the outer cannula, when the inner cannula is in the second inner cannula position the blunt distal end portion of the inner cannula is retracted into the outer cannula such that the beveled distal end portion of the outer cannula extends beyond the blunt distal end portion of the inner cannula.

In embodiments, the locking mechanism further comprises a locking member operatively connected to the proximal handle portion via a connecting member. In certain embodiments, the connecting member can include a shaft or a pin extending through the locking member. The locking member can be configured to move between a first position and a second position about to the shaft or pin, and relative to the proximal handle.

In embodiments, the locking mechanism further comprises a biasing member operatively connecting between a first end of the locking member and the proximal handle to bias the locking member towards the first position. The first position can be a locked position preventing movement of the inner cannula relative to the outer cannula and the second position is an unlocked position allowing movement of the inner cannula relative to the outer cannula.

In embodiments, the locking mechanism is configured to move from the first position to the second position upon application of a force on the first end of the locking member that is greater than a biasing force applied to the locking member via the biasing member.

In embodiments, a cannula biasing member is operatively connecting between the proximal portion of the inner cannula and a proximal end of the proximal handle, which is configured to bias the inner cannula toward the first inner cannula position. In embodiments, the inner cannula is configured to move from the first inner cannula position to the second inner cannula position upon application of a force on the distal end portion of the inner cannula that is greater than a biasing force applied to the proximal portion of the inner cannula by the cannula biasing member.

In embodiments, a second end of the locking member, opposite the first end of the locking member, further includes a formed end configured to seat within a recess in the proximal portion of the inner cannula. In certain embodiments, the formed end of the locking member can include a ridge and the recess of the inner cannula can include a circumferential groove complimentary to the ridge. In certain embodiments, the recess of the inner cannula can include a circumferential recess complimentary to a shape of the formed end of the locking member.

In embodiments, the locking mechanism is configured to prevent movement of the inner cannula in the proximal direction when the formed end of the locking member is seated within the recess of the inner cannula to prevent inadvertent exposure of the beveled cutting edge. In embodiments, the locking mechanism biasing member is configured to automatically seat the formed end of the locking member within the recess of the inner cannula when the formed end of the locking member and the recess of the inner cannula are substantially aligned.

These and other features of the locking mechanism of the subject disclosure will become more readily apparent from the following detailed description of the preferred embodiments taken in conjunction with the drawings.

### BRIEF DESCRIPTION OF THE DRAWINGS

So that those skilled in the art to which the subject disclosure appertains will readily understand how to make and use the surgical device of the subject disclosure without undue experimentation, embodiments thereof will be described in detail herein below with reference to certain figures, wherein:

FIG. 1 is a side elevational view of a cannulized surgical device, which employs the locking mechanism of the subject invention in the proximal handle assembly, showing an inner cannula in a first inner cannula position;

FIG. 2 shows the device of FIG. 1 where the inner cannula is in a second inner cannula position; and

FIG. 3 is an enlarged perspective view of the locking mechanism shown in FIG. 1.

### DETAILED DESCRIPTION OF THE PREFERRED EMBODIMENTS

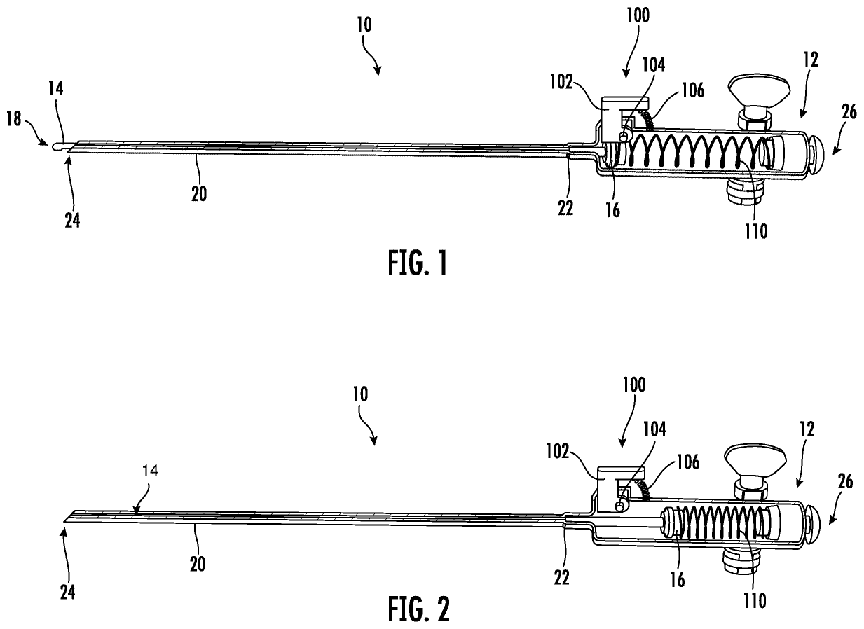

Referring now to the drawings wherein like reference numeral identify similar features or components of the subject disclosure, there is illustrated in FIG. 1, a surgical device 10 (e.g., a cannulized surgical instrument) having a proximal handle portion 12, an elongated tubular inner cannula 14 having a proximal portion 16 extending from the handle 12 and a distal end portion 18 having a first configuration and an elongated tubular outer cannula 20 having a proximal portion 22 extending from the handle 12 a distal end portion 24 having a second configuration. The inner cannula 14 is configured to move within the outer cannula 20 between a first inner cannula position (e.g., as shown in FIG. 1) and a second inner cannula position (e.g., as shown in FIG. 2), and can extend into the handle portion 12. In embodiments, e.g., as best seen in FIG. 1, the distal end portion 18 of the inner tubular cannula 14 has a blunt configuration, and, e.g., as best seen in FIG. 2, the distal end portion 24 of outer cannula 20 has cutting edge configuration to puncture a tissue (e.g., a beveled cutting edge as shown).

With reference now to FIGS. 1-3, the surgical device 10 also includes a locking mechanism 100 operatively associated with the handle 12. The locking mechanism 100 is adapted and configured to lock the inner cannula 14 relative to the outer cannula 20 to prevent movement of the inner cannula 14 between the first inner cannula position and the second inner cannula position.

The locking mechanism 100 is adapted and configured to lock the inner cannula in the first inner cannula position. When the inner cannula 14 is in the first inner cannula position, the blunt distal end portion 18 of the inner cannula extends beyond the beveled distal end portion 24 of the outer cannula 20, as shown in FIG. 1. When the inner cannula 14 is in the second inner cannula position, the blunt distal end portion 18 of the inner cannula 14 is retracted into the outer cannula 20 such that the beveled distal end portion 24 of the outer cannula 20 extends beyond the blunt distal end portion 18 of the inner cannula 14, as shown in FIG. 2. Thus, locking the inner cannula 14 in the first inner cannula position prevents inadvertent puncture of surrounding tissue by the beveled distal end portion 24 when advancing through a surgical site, such as an abdominal cavity of a patient, for example.

In embodiments, the locking mechanism 100 further comprises a locking member 102 operatively connected to the proximal handle portion 12 via a connecting member 104. In certain embodiments, the connecting member can include a shaft or a pin (e.g., as shown) extending through the locking member 102. The locking member 102 can be configured to articulate between a first articulated position and a second articulated position relative to the shaft or pin 104. For example, the locking member 102 can be configured to rocker about the shaft or pin 104 between the first and second articulated positions. Any suitable activation means is contemplated herein, for example the locking member 102 can be or include, a button, a pivoting member, or the like.

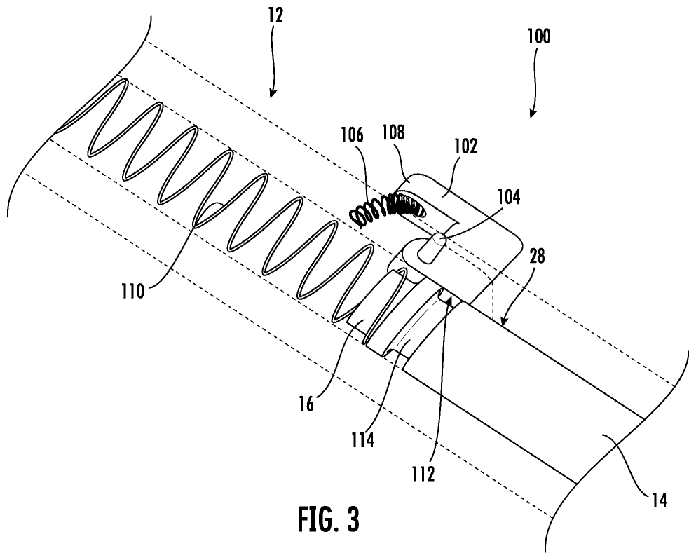

The first position of the locking member 102 can be a locked position preventing movement of the inner cannula 14 relative to the outer cannula 20 and the second position of the locking member 102 can be an unlocked position allowing movement of the inner cannula 14 relative to the outer cannula 20. In FIG. 3, the locking member 102 is shown in the first position, the locked position. In embodiments, the locking mechanism 100 further comprises a biasing member 106 operatively connecting between a first end 108 of the locking member 102 and the proximal handle 12 to bias the locking member 102 towards the first position, e.g., as shown in FIG. 3. The locking mechanism 102 is configured to move from the first position to the second position upon application of a force on the first end 108 of the locking member 102 that is greater than a biasing force applied to the locking member 102 via the biasing member 106.

In embodiments, a cannula biasing member 110 is operatively connected between the proximal portion 16 of the inner cannula 14 and a proximal end 26 of the proximal handle 12. The cannula biasing member 110 is configured to bias the inner cannula 14 toward the first inner cannula position. The inner cannula 14 is configured to move from the first inner cannula position to the second inner cannula position upon application of a force on the distal end portion 18 of the inner cannula 14 that is greater than a biasing force applied to the proximal portion 16 of the inner cannula by the cannula biasing member 110.

In embodiments, a second end 112 of the locking member 102, opposite the first end of the locking member, further includes a formed end configured to seat within a recess 114 in the proximal portion 16 of the inner cannula 14. In certain embodiments, the formed end 112 of the locking member 102 can include a ridge (e.g., as shown in FIG. 3) and the recess 114 of the inner cannula 14 can include a circumferential groove complimentary to the ridge (e.g., as shown in FIG. 3). In certain embodiments, the recess of the inner cannula can include any circumferential recess that has a complimentary to a shape of the formed end 112 of the locking member 102, and any suitable shape is contemplated herein.

In embodiments, the locking mechanism 100 is configured to prevent movement of the inner cannula 14 in the proximal direction when the formed end 112 of the locking member 102 is seated within the recess 114 of the inner cannula 14 to prevent inadvertent exposure of the beveled cutting edge 24 of the outer cannula 20. In embodiments, the locking mechanism biasing member 106 is configured to automatically seat the formed end 112 of the locking member 102 within the recess 114 of the inner cannula 14 when the formed end 112 of the locking member and 102 the recess 114 of the inner cannula 14 are substantially aligned, e.g., by biasing the locking member to the first position. When the inner cannula 14 is in the second inner cannula position (e.g., it is not locked by the locking member 102), the formed end 112 of the locking member can ride along an outer surface 28 of the inner cannula 14 until the inner cannula 14 returns to the first inner cannula position.

Provided herein is an embodiment of a locking mechanism configured to lock an inner cannula relative to an outer cannula. For example, in embodiments, the locking mechanism can be configured to lock an inner stylet (e.g., having a dull tip), relative to the outer cannula (e.g., having a sharp beveled edge) to prevent accidental or inadvertent penetration of surrounding tissue. The dull tip and beveled edge can both be at a distal end of the cannulized surgical device. The device can include a proximal portion, e.g., a proximal handle portion, being opposite the distal end. In certain embodiments, the cannulized surgical device can include a veress needle (e.g., as shown and described herein), however it is contemplated that embodiments of the locking mechanism can be included on any suitable cannulized device having a sharp outer cannula and a dull inner stylet, such as an intra-abdominal suture device, or the like.

Embodiments of the locking mechanism can include a locking member and biasing member can be included configured to bias the locking member into a locked position. The locking member can include a formed end configured to seat within a recess defined in a proximal portion of the inner stylet, to lock the inner stylet in place. For example, the formed end can include a ridge and the recess of the inner stylet can include a circumferential groove to seat the ridge, to prevent upward movement (e.g., in a proximal direction) of the inner stylet to expose the sharp beveled edge of the outer cannula. Any suitable shape for the formed end and recess are contemplated herein, so long as they are complimentary.

In operation, embodiments of the locking mechanism will hold the inner stylet in the locked position, until the locking member is depressed against the force of the biasing member (e.g. a spring or resilient member), allowing the inner stylet to freely move as needed, for example to penetrate the abdominal cavity using the sharp beveled edge of the outer cannula. In embodiment, the locking member need not be held down beyond the initial to unlocking in order to maintain the unlocked state. Once the outer cannula has successfully penetrated the desired tissue, the spring force on the inner stylet (e.g., from a cannula biasing member provided in the proximal handle) will force the inner stylet to its biased position so that the dull tip extends beyond the sharp beveled edge of the outer cannula. The biasing member of the locking mechanism then maintains a force on the locking member in the unlocked position, so that when the inner stylet returns to its biased position (e.g., the first inner cannula position), the locking member will catch the seat at the proximal end of the inner stylet, locking the inner stylet in place. Therefore, a user is not required to relock the locking mechanism. Accordingly, the locking mechanism will lock the dull tip of the inner stylet in place while the surgical device enters the body cavity, preventing any additional trauma to underlying tissue.

While the subject disclosure has been shown and described with reference to preferred embodiments, those skilled in the art will readily appreciate that changes and/or modifications may be made thereto without departing from the scope of the subject disclosure.

What is claimed is:

1. A locking mechanism for a surgical device having one or more relatively movable telescoping tubular portions configured for movement along a longitudinal axis between a first position and a second position, the locking mechanism being associated with at least one of the relatively moveable tubular portions and being configured to selectively control the relative longitudinal movement of the one or more tubular portions between the first position and the second position.

2. A surgical device, comprising:
a) a proximal handle;
b) an elongated tubular inner cannula having a proximal portion extending from the handle and a distal end portion having a first configuration;
c) an elongated tubular outer cannula having a proximal portion extending from the handle a distal end portion having a second configuration, wherein the inner cannula is configured to move within the outer cannula between a first inner cannula position and a second inner cannula position; and
d) a locking mechanism operatively associated with the handle and is adapted and configured to lock the inner cannula relative to the outer cannula to prevent movement of the inner cannula between the first inner cannula position and the second inner cannula position.

3. The device of claim 2, wherein the distal end portion of the inner tubular cannula has a blunt configuration.

4. The device of claim 2, wherein the distal end portion of outer cannula has cutting edge configuration to puncture a tissue.

5. The device of claim 2, wherein the locking mechanism is adapted and configured to lock the inner cannula in the first inner cannula position.

6. The device of claim 5, wherein when the inner cannula is in the first inner cannula position the blunt distal end portion of the inner cannula extends beyond the distal end portion of the outer cannula, and when the inner cannula is in the second inner cannula position the blunt distal end portion of the inner cannula is retracted into the outer cannula such that the distal end portion of the outer cannula extends beyond the blunt distal end portion of the inner cannula.

7. The device of claim 6, wherein the locking mechanism further comprises a locking member operatively connected to the proximal handle via a connecting member.

8. The device of claim 7, wherein the connecting member includes a shaft extending through the locking member, and wherein the locking member is configured to move between a first position and a second position about to the shaft, relative to the proximal handle.

9. The device of claim 8, wherein the locking mechanism further comprises a biasing member operatively connecting between a first end of the locking member and the proximal handle to bias the locking member towards the first position.

10. The device of claim 9, wherein the first position is a locked position preventing movement of the inner cannula relative to the outer cannula and wherein the second position is an unlocked position allowing movement of the inner cannula relative to the outer cannula.

11. The device of claim 10, wherein the locking mechanism is configured to move from the first position to the second position upon application of a force on the first end of the locking member that is greater than a biasing force applied to the locking member via the biasing member.

12. The device of claim 11, further comprising a cannula biasing member operatively connecting between the proximal portion of the inner cannula and a proximal end of the proximal handle, which is configured to bias the inner cannula toward the first inner cannula position.

13. The device of claim 12, wherein the inner cannula is configured to move from the first inner cannula position to the second inner cannula position upon application of a force on the distal end portion of the inner cannula that is greater than a biasing force applied to the proximal portion of the inner cannula by the cannula biasing member.

14. The device of claim 13, wherein a second end of the locking member, opposite the first end of the locking member, further includes a formed end configured to seat within a recess in the proximal portion of the inner cannula.

15. The device of claim 14, wherein the formed end of the locking member includes a ridge and wherein the recess of the inner cannula includes a circumferential groove complimentary to the ridge.

16. The device of claim 15, wherein the recess of the inner cannula includes a circumferential recess complimentary to a shape of the formed end of the locking member.

17. The device of claim 14, wherein the locking mechanism is configured to prevent movement of the inner cannula in the proximal direction when the formed end of the locking member is seated within the recess of the inner cannula.

18. The device of claim 17, wherein the locking mechanism biasing member is configured to automatically seat the formed end of the locking member within the recess of the inner cannula when the formed end of the locking member and the recess of the inner cannula are substantially aligned.
